(19) United States
(12) Patent Application Publication
(10) Pub. No.: US 2015/0071978 A1
(43) Pub. Date: Mar. 12, 2015

(54) CLOTHING AND COVERING SYSTEM WITH VARIOUS FUNCTIONS

(71) Applicant: Alice Chang, South Pasadena, CA (US)

(72) Inventor: Alice Chang, South Pasadena, CA (US)

(21) Appl. No.: 14/089,362

(22) Filed: Nov. 25, 2013

Related U.S. Application Data
(60) Provisional application No. 61/874,967, filed on Sep. 6, 2013.

(51) Int. Cl. A61K 9/00; A61N 2/06

(52) U.S. Cl. CPC A61K 9/0014; A61N 2/06
USPC 424/402; 600/15; 428/221

(57) ABSTRACT

Clothing and covering system with various functions comprising at least one item, having at least one layer, at least one fabric, and at least one function. At least one function is selected from the group consisting of wearing on body, fashion display, preventing snow, preventing wind, preventing sun, preventing dust and dirt, support, comfort, performance, protection, insulation, warmth, coverage, storage, containment, drying, wiping, drainage, security, housing, water flotation, thermal insulation, heat resistance, bullet proofing, sun screening, non ironing, breasts shaping, arch support, seamless footwear, bandaging, cover polarizing, pressure addition, cover planting, electricity generation, food storage with coated paper, odor absorption, aromatherapy, moisturizing, cleansing, dust trapping, glow-in-the-dark, water and leakage proofing, and ingredient addition. Leakproof clothing and covering can control and prevent both leakage of body discharge and secretion, and leakage of rain and water. Clothing and covering with ingredient addition includes flavoring ingredient, cleansing ingredient, nutritional ingredient, and health ingredient.

### CROSS-REFERENCE TO RELATED APPLICATION

The present application is related to and claims priority from prior provisional application Ser. No. 61/874,967 filed Sep. 6, 2013 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### FIELD OF THE INVENTION

The present invention relates generally to the field of garments and more specifically relates to a clothing and covering system with various functions

### DESCRIPTION OF THE RELATED ART

Clothing and covering are typically fiber and textile material. Clothing can be worn as a covering on the body of a wearer. The wearing of clothing is a feature of nearly all modern human societies. The amount and type of clothing worn may depend on physical, social and geographic considerations. Clothing worn normally serves as a fashionable covering and as an insulator from ambient conditions. Other kinds of clothing include cloth, towel, pad, and so on. Covering can be used to cover and contain objects. And covering can be in the form of cover, screen, shield, shade, blind, curtain, carpet, mat, bag, box, container, bandage, and so on. Clothing and covering are not traditionally multi-purpose in design.

Clothing and covering can have leakage of discharge and secretion of the body. Discharge and secretion of the body include perspiration, urine, semen, menstrual blood, feces, and body oils. Leakage of discharge and secretion of the body causes the clothing and covering to stain and smell, and causes embarrassments. Clothing and covering can have leakage of rain and water. Leakage of rain and water causes the clothing and covering to get wet. Clothing and covering on the market is created without any ingredient, without flavoring ingredient, cleansing ingredient, nutritional ingredient, and health ingredient. Without flavoring ingredient, the clothing and covering do not have a fresh scent. Without cleansing ingredient, the clothing and covering are more likely to be contaminated and emit odors. Without nutritional ingredient, the wearer of the clothing and covering does not get essential nutrients. Without health ingredient, the wearer of the clothing and covering does not get health benefits. Other functions are also missing from clothing and covering.

Various attempts have been made to solve the above-mentioned problems such as those found in U.S. Pat. No. 7,993,322 to Lynn Brud; U.S. Pat. No. 4,555,245 to Thomas L. Armbruster; U.S. Pat. No. 6,591,425 to Mary P. Zellers; U.S. Publication No. 2006/0168704 to Judy Mayer and David Mayer; U.S. Pat. No. 6,347,407 to Brenda Diane Dowell; and U.S. Publication No 2008/0052802 to Laura Bryan. This art is representative of garments. None of the above inventions and patents, taken either singly or in combination, is seen to describe the invention as claimed.

Ideally, clothing and covering system can control and prevent both leakage of discharge and secretion of the body, and leakage of rain and water. Clothing and covering system with ingredient addition include flavoring ingredients, cleansing ingredients, nutritional ingredients, and health ingredient. So that clothing and covering can have a fresh scent, and is less likely to be contaminated and emit odors. Further, wearer of the clothing and covering can get essential nutrients, and wearer of the clothing and covering can get health benefits. Clothing and covering system can also comprise other functions. Clothing and covering system would operate reliably and be manufactured at a modest expense. Thus, a need exists for clothing and covering system to avoid the above mentioned problems.

### BRIEF SUMMARY OF THE INVENTION

In view of the foregoing disadvantages inherent in the known clothing and covering art, the present invention provides a novel clothing and covering system with various functions.

A clothing and covering system is disclosed herein, in a preferred embodiment, comprising: clothing and covering, having at least one layer, at least one fabric, and at least one function.

At least one function is selected from the group consisting of wearing on body, fashion display, preventing snow, preventing wind, preventing sun, preventing dust and dirt, support, comfort, performance, protection, insulation, warmth, coverage, storage, containment, drying, wiping, drainage, security, housing, water flotation, thermal insulation, heat resistance, bullet proofing, sun screening, non ironing, breasts shaping, arch support, seamless footwear, bandaging, cover polarizing, pressure addition, cover planting, electricity generation, food storage with coated paper, odor absorption, aromatherapy, moisturizing, cleansing, dust trapping, glow-in-the-dark, water and leakage proofing, and ingredient addition.

A method of use for the clothing and covering system is disclosed herein preferably comprising the steps of: donning at least one item; using at least one item; and removing at least one item after duration of time.

The present invention holds significant improvements and serves as a novel clothing and covering system with various functions. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### DETAILED DESCRIPTION

As discussed above, embodiments of the present invention relate to a novel clothing and covering system with various functions, including leakproof clothing and covering, clothing and covering with ingredient addition, and clothing and covering with other functions.

As discussed above, the clothing and covering system with various functions comprises clothing and covering, having at least one layer, at least one fabric, and at least one function.

Clothing and covering including T-shirt, panty, bra, brief, boxer, short, underpant, long john, bikini, thong, G-string, jacket, parka, coat, blazer, trench, shirt, sleeveless shirt, polo shirt, cardigan, sweater, sweatshirt, hoodie, pant, trouser, jean, suit, tuxedo, vest, tie, bow tie, handkerchief, scarf, muffler, dress, skirt, short, capri, gown, cape, cloak, robe, mantle, poncho, shawl, shroud, stole, face mask, veil, hat, cap, helmet, flag, badge, patch, handband, wristband, armband, elbowband, ankleband, kneeband, footband, legband, waistband, glove, sock, panty hose, legging, tight, leotard, night gown, lingerie, apron, bottom, top, towel, paper towel, kitchen towel, bathroom towel, dishcloth, dustcloth, air filter, cloth, bandage, pad, belt, bottle, feeding bottle, spray bottle, pump bottle, container, paper container, plastic container, metal container, thermal container, pressurized container, food container, drink container, cosmetics container, fragrance container, moisturizer container, cleanser container, soap dispenser, tube, box, paper box, plastic box, metal box, thermal box, lunchbox, jewelry box, watch box, eyewear box, wallet, purse, key holder, backpack, handbag, duffel bag, satchel bag, messenger bag, sling bag, golfbag, laptop bag, bag, tote, sack, suitcase, briefcase, blanket, quilt, pillow, cushion, comforter, duvet cover, sham, pillow case, bed sheet, mattress cover, mattress pad, seating pad, tablecloth, linen, bedding, furniture covering, computer covering, keyboard cover, cell phone covering, mousepad, pad, carpet, rug, mat, doormat, bathmat, placemat, car mat, cover, car cover, vehicle cover, car seat cover, sofa cover, couch cover, cushion cover, paint, wall, ceiling, roof, floor, wall cover, ceiling cover, floor cover, shade, shield, blind, door, plastic door, glass door, metal door, window, plastic window, glass window, metal window, car window, window shade, window blind, shutter, screen, window screen, TV screen, computer screen, cell phone screen, curtain, shower curtain, umbrella, eyeglass, sunglass, tent, sleeping bag, punching bag, sandbag, air bag, tea bag, popcorn bag, lunch bag, paper bag, plastic bag, grocery bag, shopping bag, gunny sack, thermal bag, body bag, burn bag, money bag, mail bag, antistatic bag, bulk bag, disposable bag, reusable bag, garbage bag, airsickness bag, zipper bag, drawstring bag, string bag, diaper bag, mosquito net, car cover, shoe, boot, sandal, slipper, sneaker, skate, lens, eyeglass, sunglass, uniform, armor, ballistic vest, bulletproof vest, bombsuit, life jacket, firesuit, protective suit, spacesuit, pressure suit, baselayer, underwear, lounge wear, brassiere, hosiery, daywear, evening wear, formal wear, bridal wear, outerwear, earwear, eyewear, headwear, footwear, dance suit, swimsuit, diving suit, track suit, skiwear, snowwear, rainwear, beach wear, activewear, sportswear, vehicle, building, and so on.

Clothing and covering having feature selected from the group consisting of fabric disclosure, washing instruction, brand label, printed label, label, sign, patch, badge, stamp, tag, hood, pocket, collar, collar stay, shirt stay, lapel, neckline, turtle neck, mock neck, crew neck, funnel neck, cowl neck, jewel neck, boat neck, single breast, double breast, placket, pleat, fold, roll, ruffle, bubble, crop, sleeve, raglan, tail, cuff, hem, fly, gusset, corset, cleavage, thread, fastener, closure, knob, latch, frame, panel, rail, notion, seam, twill, button, button hole, hole,

vent, air vent, pom pom, tassel, strap, buckle, belt, band, elastic band, tape, resealable tape, velcro, hook, eye, zipper, rivet, grommet, snap, seal, pin, safety pin, screw, screw cap, cap, bottle cap, safety cap, stay tab, pull tab, lid, cork, staple, slide, clasp, tack, nail, clip, knot, loop, string, drawstring, whip, rod, toggle, suspender, rope, tie, tape, handle, strap, flap, hinge, lock, wheel, chain, wire, underwire, ring, heel, shoelace, shank, insole, outsole, crampon, cleat, stud, welt, tongue, aglet, glue, ribbon, cord, shoulder pad, pad, gauze, film, mesh, wool, foam, gel, corduroy, crochet, velvet, velveteen, velour, satin, lace, cable, pique, honeycomb, sequin, chiffon, fleece, flannel, felt, denim, gabardine, herringbone, paisley, shield, shade, cover, cloth, towel, terry, braid, twist, net, canvas, needlework, quilt, embroidery, tapestry, upholstery, pattern, print, dye, color, lens, glass, ornament, jewelry, brooch, cuff link, corrugated fiberboard, paperboard, covered elastic band, covered hood under collar, silicon pad, stone washing, non ironing, preshrunk, seamless, stitching, sewing, knitting, weaving, quilting, crafting, tailoring, molding, shaping, cutting, opening, trimming, piping, beading, webbing, facing, backing, lining, filing, layering, pressure, oxygen, spray, pump, wicking reed, candle, wax, wick, fragrance, aroma, deodorant, moisturizer, cleanser, glow, filter, plant, coating, paint, polish, solar panel, drainage, slope, architecture, design, construction, electrical, plumbing, telecommunication, heating, ventilation, air conditioning, water, gas, lighting, escalator, elevator, blister pack, skin pack, child resistant packaging, and so on.

Clothing and covering including category selected from the group consisting of rainwear, snowwear, skiwear, hospital, military, police, firefighting, lifeguard, space, flight, diving, protection wear, uniform, cleaning, kitchen, bathroom, bedding, brassiere, hosiery, eyewear, head wear, underwear, lounge wear, daywear, eveningwear, couture, casual wear, business wear, formal wear, bridal wear, beachwear, outerwear, footwear, activewear, sportswear, outdoor, towel, cloth, pad, cover, shade, shield, screen, blind, bandage, bag, box, bottle, container, food container, drink container, cosmetics container, fragrance container, moisturizer container, cleanser container, soap dispenser, luggage, curtain, carpet, door, window, wall, ceiling, floor, car window, window covering, floor covering, wall covering, ceiling covering, roof covering, solar panel, furniture, air filter, umbrella, lens, eyeglass, sunglass, computer, cell phone, vehicle, vehicle covering, building, and so on.

At least one layer comprising on surface selected from the group consisting of color, pattern, figure, drawing, writing, sign, label, logo, and advertisement. At least one layer is selected from the group consisting of disposable, replaceable, washable, and reusable.

At least one fabric is selected from the group consisting of waterproof fabric, synthetic fabric, natural fabric, and elastic fabric.

At least one function is selected from the group consisting of wearing on body, fashion display, preventing snow, preventing wind, preventing sun, preventing dust and dirt, support, comfort, performance, protection, insulation, warmth, coverage, storage, containment, drying, wiping, drainage, security, housing, water flotation, thermal insulation, heat resistance, bullet proofing, sun screening, non ironing, breasts shaping, arch support, seamless footwear, bandaging, cover polarizing, pressure addition, cover planting, electricity generation, food storage with coated paper, odor absorption, aromatherapy, moisturizing, cleansing, dust trapping, glow-in-the-dark, water and leakage proofing, and ingredient addition.

At least one layer has addition of at least one ingredient selected from the group consisting of flavoring ingredient, cleansing ingredient, nutritional ingredient, and health ingredient. At least one ingredient can be delivered without using adhesive on the skin. At least one ingredient can also be delivered by using adhesive on the skin. At least one ingredient can be delivered to a large surface area of body. At least one ingredient can be delivered to specific part of the body. At least one ingredient is added to at least one layer selected from the group consisting of specific section of at least one layer, and entire at least one layer. At least one ingredient is in the form selected from the group consisting of extract, juice, sauce, liquid, water, oil, alcohol, lotion, gel, wax, jelly, paste, powder, and small pieces. At least one ingredient is added to at least one layer by method selected from the group consisting of imbedded, coated, sewn, and glued. And at least one ingredient can be contained in the container with at least one layer.

At least one fabric has addition of at least one ingredient selected from the group consisting of flavoring ingredient, cleansing ingredient, nutritional ingredient, and health ingredient. At least one ingredient is in the form selected from the group consisting of extract, juice, sauce, liquid, water, oil, alcohol, lotion, gel, wax, jelly, paste, powder, and small pieces. At least one ingredient is added to at least one fabric by method selected from the group consisting of imbedded, coated, sewn, and glued.

At least one fabric comprises waterproof fabric selected from the group consisting of natural fabric, and synthetic fabric. The natural fabric includes shell, pearl, mineral, metal, rubber, resin, and wax. The synthetic fabric includes plastic, bio-plastic, latex, nylon, polyester, rayon, acrylic, acetate, lastex, kevlar, gore-tex, polyvinyl chloride (PVC), polyurethane (PU), silicone elastomer, fluoropolymers, polyamide, polypropylene, polyethylene, polyethylene terephthalate, microfiber, polymer, fiberglass, glass, concrete, cement, brick, stucco, plaster, paint, resin, geopolymer, geotextile, ultrasuede, silicone, olefin, ingeo, lurex, nomex, marian, viscose, aramid, mineral wax, petroleum wax, and synthetic wax. The waterproof fabric can be interwoven with elastic fabric for clothing stretchability.

At least one fabric comprises synthetic fabric selected from the group consisting of plastic, bio-plastic, latex, nylon, polyester, rayon, acrylic, modacrylic, acetate, lastex, kevlar, gore-tex, polyvinyl chloride (PVC), polyurethane (PU), silicone elastomer, fluoropolymers, polyamide, polypropylene, polyethylene, polyethylene terephthalate, styrofoam, microfiber, polymer, fiberglass, glass, concrete, cement, brick, stucco, plaster, paint, resin, geopolymer, geotextile, ultrasuede, silicone, olefin, ingeo, lurex, nomex, marian, viscose, aramid, mineral wax, petroleum wax, and synthetic wax. The synthetic fabric can be interwoven with elastic fabric for clothing stretchability.

At least one fabric comprises natural fabric selected from the group consisting of plant fiber, vegetable fiber, wood fiber, animal fiber, metallic fiber, carbon fiber, and mineral fiber, including cotton, silk, linen, flax, jute, sisal, ramie, tencel, wool, cashmere, alpaca, angora, llama, mohair, pashmina, camel hair, spider silk, bamboo, hemp, wood, paper, down, feather, leather, suede, shearling, fur, skin, hair, bone, horn, shell, pearl, mineral, stone, rock, soil, sand, clay, metal, rubber, resin, plant wax, and animal wax. The natural fabric has ability to absorb moisture and is comfortable to wear. The natural fabric can be interwoven with synthetic fabric for clothing durability. The natural fabric can be interwoven with elastic fabric for clothing stretchability. The natural fabric can be interwoven with both synthetic fabric and elastic fabric.

At least one fabric comprises elastic fabric selected from group consisting of spandex, elastane, and lycra. The elastic fabric has ability to stretch.

At least one function comprises water flotation which provides buoyancy for flotation in water by air inflation. The fabric used for air inflation includes two layers of bonded polyurethane.

At least one function comprises thermal insulation which maintains temperature of content to stay cold and stay hot. The thermal insulation using fabric selected from the group consisting of PET film, styrofoam, polyethylene, polyurethane, and polypropylene.

At least one function comprises heat resistance which provides insulation from heat and fire. The heat resistance using fabric selected from the group consisting of marian, fiberglass, and modacrylic.

At least one function comprises bullet proofing which provides capability of stopping a bullet and similar high velocity projectile. The bullet proofing using fabric selected from the group consisting of kevlar, lexan, polyethylene, aramid, ceramic, metal, and carbon fiber composite.

At least one function comprises sun screening which provides sun protection, and sun protective clothing is produced from a fabric rated for its level of ultraviolet (UV) protection. The sun screening using fabric selected from the group consisting of fabric with a novel weave structure and denier, and fabric pre-treated with ultraviolet inhibiting ingredient including dye, titanium dioxide, and zinc oxide.

At least one function comprises non ironing with coating for permanent press which suppresses wrinkles and creates a smooth appearance without ironing. The permanent press using chemical selected from the group consisting of polytetrafluoroethylene, formaldehyde, urea-formaldehyde derivative, and urea derivative.

At least one function comprises breasts shaping which provides breasts with shaping of fit and size by method selected from the group consisting of using bra underwire and padding. The bra underwire uses a thin, semi-circular strip of rigid material fitted inside bra fabric under each cup. The material for bra underwire includes metal, plastic, and resin. The padding uses a pad for each cup. The material for padding includes natural fabric, synthetic fabric, and silicon gel.

At least one function comprises arch support which provides support for arch of foot by method selected from the group consisting of placing a piece of material on top of insole of shoe having molded form fitting arch of foot, and making insole of shoe with material having molded form fitting arch of foot. The material is selected from the group consisting of natural fabric, synthetic fabric, plastic, bio-plastic, rubber, and silicon gel.

At least one function comprises seamless footwear which provides comfort for foot by using sock like seamless material in one piece to wrap foot for footwear. The seamless footwear is selected from the group consisting of single layer, multiple layer, inner layer, middle layer, and outer layer. The footwear is selected from the group consisting of shoe, boot, slipper, sneaker, skate, ski footwear, swim footwear, and diving footwear. The seamless material can be imbedded with material selected from the group consisting of activated charcoal, baking soda, and magnets. The seamless material is selected from the group consisting of disposable, replaceable, and reusable. The seamless material is selected from the group consisting of natural fabric, synthetic fabric, plastic, bio-plastic, rubber, and silicon gel.

At least one function comprises bandaging which provides support and dressing to specific part of body by method selected from the group consisting of wrapping dressing without adhesive, wrapping dressing with adhesive, wrapping splint without adhesive, and wrapping splint with adhesive. The dressing is selected from the group consisting of cloth, gauze, film, gel, foam, hydrocolloid, alginate, hydrogel, and polysaccharide paste, granule, and bead. The splint is selected from the group consisting of fiberglass, and orthoglass.

At least one function comprises cover polarizing which provides reduction of glare by using polarizer and polarizing filter. The cover polarizing is for cover selected from the group consisting of eyewear, window, car window, door, shade, shield, screen, and curtain. The cover polarizing using fabric selected from the group consisting of synthetic plastic sheet including polyvinyl alcohol (PVA) polymer impregnated with iodine, and aligned polyvinylene chains.

At least one function comprises pressure addition which provides suit wearer with pressurized oxygen within the sealed suit. The pressure addition is useful for suit selected from the group consisting of space suit, flight suit, diving suit, ski suit, and pressure suit.

At least one function comprises cover planting which provides cover with planting. The cover planting is for cover selected from the group consisting of wall, floor, ceiling, roof, window, door, shade, shield, screen, curtain, and carpet. The cover can be partially and completely covered by irrigated living plant. The cover planting is selected from the group consisting of loose medium, mat medium, and structural medium. The cover planting can provide environmental, social, and economic benefits.

At least one function comprises electricity generation which provides generation of electricity with installation of solar panels. The solar panels are installed on top of the clothing and covering, including roof, wall, building, and vehicle.

At least one function comprises food storage with coated paper which provides paper container and carton with ability of food storage. The food storage with coated paper can store liquid food and dry food. The paper container and carton using coating selected from the group consisting of plant wax, animal wax, beeswax, carnauba wax, paraffin wax, polyethylene, aluminum foil, and bio-plastic.

At least one function comprises odor absorption which provides absorption of odor from atmosphere. The odor absorption using container storing material selected from the group consisting of activated charcoal, baking soda, zeolite, silica gel, oxidizing agent, air sanitizer, antiseptic, deodorant, and disinfectant. The material is in form selected from the group consisting of solid powder, solid particles, liquid, gel, wax, and jelly. The container selected from the group consisting of cloth, paper, plastic, glass, and metal. The container is in form selected from the group consisting of porous container, and non porous container with top opening. The porous container is in form selected from the group consisting of box, bottle, and bag. The box having edge supporting perimeter frame and porous layer on one to six sides for surface area exposure of enclosed material.

At least one function comprises aromatherapy which provides treatment and prevention of disease by use of fragrant oil, fragrant water, fragrant alcohol, fragrant wax, and fragranced material. The aromatherapy having mode of application selected from the group consisting of aerial diffusion, direct inhalation, and topical application. The aerial diffusion is by method selected from the group consisting of vaporization, heat application, use of spray bottle, use of wicking reed, use of fragrant drawer paper and drawer cloth, use of fragrant botanical in porous container and cloth container, and burning of fragrant candle. The direct inhalation is by method selected from the group consisting of inhaling from small container with fragrant liquid, and using fragranced face mask. The topical application is by method selected from the group consisting of use of clothing and covering, including spray bottle, pump bottle, container, and dressing.

At least one function comprises moisturizing which provides moisturizer selected from group consisting of plant oil, vegetable oil, mineral oil, jojoba oil, vegetable fat, animal fat, plant wax, animal wax, beeswax, carnauba wax, paraffin wax, aloe vera, petroleum jelly, collagen, snail slime, and antioxidant. The moisturizing having mode of application consisting of topical application. The topical application is by method consisting of use of clothing and covering, including spray bottle, pump bottle, container, and dressing.

At least one function comprises cleansing which provides cleanser by use of cleansing ingredient selected from the group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, benzoyl peroxide, resorcinol, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium tetrachlorohydrex gly, activated charcoal, zeolite, silica gel, oxidizing agent, air sanitizer, antiseptic, deodorant, and disinfectant. The cleansing having mode of application consisting of topical application, wiping, and washing. The mode of application is by method consisting of use of clothing and covering, including spray bottle, pump bottle, container, and dressing.

At least one function comprises dust trapping which provides the ability to trap dust particles. The dust trapping is by method consisting of use of said clothing and covering, including dustcloth, air filter, screen, and face mask. The dust trapping using material selected from the group consisting of tack, wax, oil, polyester, paper, and cloth. The material is in form selected from the group consisting of mesh, woven, pleated, layered, and textured.

At least one function comprises glow-in-the-dark which provides the ability to glow in the dark. The glow-in-the-dark is selected from the group consisting of phosphorescence, and fluorescence. The phosphorescence using material selected from the group consisting of zinc sulfide, and strontium aluminate. The fluorescence using material selected from the group consisting of vitamin B2, quinine, pyranine, gemstone, and mineral.

At least one function comprises water and leakage proofing which controls and prevents both leakage of discharge and secretion of body, and leakage of rain and water. The water and leakage proofing can be accomplished by leakproof clothing and covering. The leakproof clothing and covering comprise at least one fabric selected from the group consisting of waterproof fabric, synthetic fabric, natural fabric, and elastic fabric. The waterproof fabric of the 1st kind is not interwoven with other fabric. The waterproof fabric of the 2nd kind is interwoven with elastic fabric. The natural fabric of the 1st kind is not interwoven with other fabric. The natural fabric of the 2nd kind is interwoven with synthetic fabric. The natural fabric of the 3rd kind is interwoven with elastic fabric. The natural fabric of the 4th kind is interwoven with both synthetic fabric and elastic fabric.

At least one function comprises ingredient addition which provides addition of at least one ingredient selected from the group consisting of flavoring ingredient, cleansing ingredient, nutritional ingredient, and health ingredient.

Leakproof clothing and covering can be used in conjunction with disposable including sanitary pad, sanitary napkin, panty liner, tampon, incontinence pad, diaper, underarm liner, and so on. The disposable comprises material selected from the group consisting of adhesive, resealable tape, elasticised waist band, natural material, synthetic material, superabsorbent polymer, oil, gel, wax, jelly, lotion, fragrance, and so on. The disposable can be placed and glued to specific area of the leakproof clothing and covering, including underarm area, foot area, hand area, neck area, head area, crotch area, groin area, genital area, and so on.

Leakproof clothing and covering has 26 kinds:

1. The leakproof clothing and covering of the 1st kind is created with a single layer of waterproof fabric. The wearer can have the option to wear a garment underneath the leakproof clothing and covering. And the garment can be made of natural fabric.
2. The leakproof clothing and covering of the 2nd kind is created with a single layer of natural fabric, with an outer coating of waterproof fabric, and an inner side has natural fabric.
3. The leakproof clothing and covering of the 3rd kind is created with a single layer of natural fabric, with an inner coating of waterproof fabric, and an outer side has natural fabric.
4. The leakproof clothing and covering of the 4th kind is created with a single layer of natural fabric, with an inner coating of waterproof fabric, and an outer coating of waterproof fabric.
5. The leakproof clothing and covering of the 5th kind is created by having 2 layers, with an inner layer of natural fabric, and an outer layer of waterproof fabric.
6. The leakproof clothing and covering of the 6th kind is created by having 2 layers, with an outer layer of natural fabric, and an inner layer of waterproof fabric.
7. The leakproof clothing and covering of the 7th kind is created with a single layer of natural fabric, having section made of waterproof fabric as an outer coating, and an inner side has natural fabric. The section includes underarm area, crotch area, groin area, genital area, upper torso of the body, lower torso of the body, trunk of the body, feet, hand, neck, head, and so on.
8. The leakproof clothing and covering of the 8th kind is created with a single layer of natural fabric, having section made of waterproof fabric as an inner coating, and an outer side has natural fabric. The section includes underarm area, crotch area, groin area, genital area, upper torso of the body, lower torso of the body, trunk of the body, feet, hand, neck, head, and so on.
9. The leakproof clothing and covering of the 9th kind is created with a single layer of natural fabric, having section made of waterproof fabric as an inner coating, and an outer coating of waterproof fabric. The section includes underarm area, crotch area, groin area, genital area, upper torso of the body, lower torso of the body, trunk of the body, feet, hand, neck, head, and so on.
10. The leakproof clothing and covering of the 10th kind is created with a single layer of at least one fabric, having section made of waterproof fabric, and other area of the single layer made of natural fabric. The section includes underarm area, crotch area, groin area, genital area, upper torso of the body, lower torso of the body, trunk of the body, feet, hand, neck, head, and so on.
11. The leakproof clothing and covering of the 11th kind is created by having 2 layers, having section made of waterproof fabric as an outer layer, and an inner layer of natural fabric. The section includes underarm area, crotch area, groin area, genital area, upper torso of the body, lower torso of the body, trunk of the body, feet, hand, neck, head, and so on.
12. The leakproof clothing and covering of the 12th kind is created by having 2 layers, having section made of waterproof fabric as an inner layer, and an outer layer of natural fabric. The section includes underarm area, crotch area, groin area, genital area, upper torso of the body, lower torso of the body, trunk of the body, feet, hand, neck, head, and so on.
13. The leakproof clothing and covering of the 13th kind is created by having a middle layer of waterproof fabric between 2 layers of natural fabric.
14. The leakproof clothing and covering of the 14th kind is created by having a middle layer of waterproof fabric between 2 layers of waterproof fabric.
15. The leakproof clothing and covering of the 15th kind is created by having a middle layer of waterproof fabric between 2 layers, with an inner layer of natural fabric, and an outer layer of waterproof fabric.
16. The leakproof clothing and covering of the 16th kind is created by having a middle layer of waterproof fabric between 2 layers, with an outer layer of natural fabric, and an inner layer of waterproof fabric.
17. The leakproof clothing and covering of the 17th kind is created by having a middle layer of natural fabric between 2 layers of waterproof fabric.
18. The leakproof clothing and covering of the 18th kind is created by having a middle layer of natural fabric between 2 layers of natural fabric, with an outer layer having an outer coating of waterproof fabric.
19. The leakproof clothing and covering of the 19th kind is created by having a middle layer of natural fabric between 2 layers of natural fabric, with an inner layer having an outer coating of waterproof fabric.
20. The leakproof clothing and covering of the 20th kind is created by having a middle layer of natural fabric between 2 layers of natural fabric, with an outer layer having an outer coating of waterproof fabric, and an inner layer having an outer coating of waterproof fabric.
21. The leakproof clothing and covering of the 21st kind is created by having a middle layer of natural fabric between 2 layers, with an inner layer of natural fabric, and an outer layer of waterproof fabric.
22. The leakproof clothing and covering of the 22nd kind is created by having a middle layer of natural fabric between 2 layers, with an outer layer of natural fabric, and an inner layer of waterproof fabric.
23. The leakproof clothing and covering of the 23rd kind is created by having said at least one layer of waterproof fabric.
24. The leakproof clothing and covering of the 24th kind is created by having at least one coating of waterproof fabric.
25. The leakproof clothing and covering of the 25th kind is created by having at least one section of at least one layer made of waterproof fabric. The section includes underarm area, crotch area, groin area, genital area, upper torso of the body, lower torso of the body, trunk of the body, feet, hand, neck, head, and so on.
26. The clot leakproof clothing and covering of the 26th kind is created by having at least one section of at least one layer having at least one coating of waterproof fabric. The section includes underarm area, crotch area, groin area, genital area, upper torso of the body, lower torso of the body, trunk of the body, feet, hand, neck, head, and so on.

For the clothing and covering system, the flavoring ingredient can provide smell of flavor, fresh scent, aroma, and fragrance.

The flavoring ingredient is selected from the group consisting of natural flavor, artificial flavor, fruit flavor, plant flavor, herb flavor, tea flavor, root flavor, flower flavor, flower bud flavor, pollen flavor, leaf flavor, stem flavor, stem shoot flavor, bulb flavor, nut flavor, seed flavor, vegetable flavor, bean flavor, cereal grain flavor, plant oil flavor, vegetable oil flavor, vegetable fat flavor, vegetable skin flavor, fruit skin flavor, and other flavor, including artificial sweetener, salt, vinegar, sugar, syrup, honey, milk, yogurt, butter, margarine, cream, sour cream, peanut butter, butterscotch, caramel, cheese, chocolate, coffee, roasted cereal grain beverage, roasted nut and seed beverage, roasted bean beverage, malted drink, flower tea, root tea, herbal tea, mushroom tea, green tea, oolong tea, black tea, white tea, yellow tea, earl grey, darjeeling, hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litsea cubeba, marjoram, melaleuca, mountain savory, mugwort, myrrh, myrtle, neroli, nutmeg, orris, palo santo, petitgrain, pine, ravensara, red cedar, rosewood, sage, sassafras, schisandra, spikenard, spruce, tea tree, tsuga, valerian, yarrow, ylang ylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiño, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizards tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, mint, acai, aloe vera, arnica, ashoka, asthma weed, astragalus, barberry, belladonna, bitter gourd, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, watercress, wattleseed, wild betel, willow herb, wood avens, woodruff, and so on.

For the clothing and covering system, the cleansing ingredient can eliminate bacteria and odor, and clothing and covering with cleansing ingredient is less likely to contaminate and emit odor.

The cleansing ingredient is selected from the group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium tetrachlorohydrex gly, activated charcoal, zeolite, silica gel, oxidizing agent, air sanitizer, antiseptic, deodorant, disinfectant, and so on.

For the clothing and covering system, the nutritional ingredient can provide essential nutrient.

The nutritional ingredient is selected from the group consisting of essential nutrient including vitamin, dietary mineral, amino acid, and fatty acid. The vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K. The dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium. The amino acid includes histidine, isoleucine, leucine, lysine, methionine, cysteine, phenylalanine, tyrosine, threonine, tryptophan, and valine. The fatty acid includes alpha-linoleic acid (an omega-3 fatty acid) and linoleic acid (an omega-6 fatty acid).

For the clothing and covering system, the health ingredient can provide health benefit.

The health ingredient is selected from the group consisting of magnet, and medication. The magnet produces magnetic therapy. Magnetic energy is enabling people to relieve pain, reduce inflammation, produce an alkaline effect, fight infection, support healing, reduce fluid retention, increase cellular oxygen, encourage deep restorative sleep, produce a bright mental effect, reduce fatty deposits, establish healing polarity, stimulate melatonin production, and normalize natural alkaline pH. The medication can be absorbed into the skin and bloodstream.

Clothing and covering system with magnet as health ingredient provides wearer of the clothing with an easy and comfortable way to undergo magnetic therapy. The North Pole of the magnet is directly facing the body of wearer for maximum benefit. Magnet strength is measured in units called Gauss. Strength of the magnet is preferably 1200 Gauss as actual reading with a Gauss meter, and other strengths can also be used. Therapeutic magnet may vary from about 100 Gauss to 2,500 Gauss as actual reading with a Gauss meter. The magnet can be in the form of small round discs about 1 to 3 millimeters in thickness, and about 5 to 30 millimeters in diameter. Other shapes and sizes can also be used. Therapeutic magnet include ceramic magnet, neodymium magnet, samarium cobalt magnet, flexible rubber magnet, and other forms of magnets. Magnets may be imbedded, coated, sewn, or glued into the clothing.

Clothing and covering system with medication as health ingredient provides wearer of the clothing a medium to absorb medication through the skin. The clothing can provide a large surface area compared to skin patch and transdermal patch. Skin patch is a medicated adhesive patch that is placed on the skin to deliver a medication into the skin. Transdermal patch is a medicated adhesive patch that is placed on the skin to deliver a specific dose of medication through the skin and into the bloodstream. Medication is applied onto the clothing in order to deliver medication into the skin. And medication is applied onto the clothing in order to deliver medication into the bloodstream. The clothing can be worn comfortably on the wearer as compared to uncomfortable adhesive patch. The medication can be delivered from the clothing without the use of adhesive on the skin. The medication can also be delivered from the clothing with the use of adhesive on the skin. Clothing system with medication can deliver medication to a large surface area of the body, and to specific part of the body of wearer. The medication is applied to area selected from the group consisting of specific section of clothing and covering, and entire clothing and covering. The medication is in the form selected from the group consisting of extract, liquid, water, oil, alcohol, lotion, gel, wax, jelly, paste, powder, and small pieces. And the medication is applied onto the clothing by method selected from the group consisting of imbedded, coated, sewn, and glued.

The clothing and covering system with various functions is created for animal, human, infant, children, and adult; and in all sizes.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A clothing and covering system comprising: clothing and covering, having at least one layer, at least one fabric, and at least one function;
wherein said clothing and covering including T-shirt, panty, bra, brief, boxer, short, underpant, long john, bikini, thong, G-string, jacket, parka, coat, blazer, trench, shirt, sleeveless shirt, polo shirt, cardigan, sweater, sweatshirt, hoodie, pant, trouser, jean, suit, tuxedo, vest, tie, bow tie, handkerchief, scarf, muffler, dress, skirt, short, capri, gown, cape, cloak, robe, mantle, poncho, shawl, shroud, stole, face mask, veil, hat, cap, helmet, flag, badge, patch, handband, wristband, armband, elbowband, ankleband, kneeband, footband, legband, waistband, glove, sock, panty hose, legging, tight, leotard, night gown, lingerie, apron, bottom, top, towel, paper towel, kitchen towel, bathroom towel, dishcloth, dustcloth, air filter, cloth, bandage, pad, belt, bottle, feeding bottle, spray bottle, pump bottle, container, paper container, plastic container, metal container, thermal container, pressurized container, food container, drink container, cosmetics container, fragrance container, moisturizer container, cleanser container, soap dispenser, tube, box, paper box, plastic box, metal box, thermal box, lunchbox, jewelry box, watch box, eyewear box, wallet, purse, key holder, backpack, handbag, duffel bag, satchel bag, messenger bag, sling bag, golfbag, laptop bag, bag, tote, sack, suitcase, briefcase, blanket, quilt, pillow, cushion, comforter, duvet cover, sham, pillow case, bed sheet, mattress cover, mattress pad, seating pad, tablecloth, linen, bedding, furniture covering, computer covering, keyboard cover, cell phone covering, mousepad, pad, carpet, rug, mat, doormat, bathmat, placemat, car mat, cover, car cover, vehicle cover, car seat cover, sofa cover, couch cover, cushion cover, paint, wall, ceiling, roof, floor, wall cover, ceiling cover, floor cover, shade, shield, blind, door, plastic door, glass door, metal door, window, plastic window, glass window, metal window, car window, window shade, window blind, shutter, screen, window screen, TV screen, computer screen, cell phone screen, curtain, shower curtain, umbrella, eyeglass, sunglass, tent, sleeping bag, punching bag, sandbag, air bag, tea bag, popcorn bag, lunch bag, paper bag, plastic bag, grocery bag, shopping bag, gunny sack, thermal bag, body bag, burn bag, money bag, mail bag, antistatic bag, bulk bag, disposable bag, reusable bag, garbage bag, airsickness bag, zipper bag, drawstring bag, string bag, diaper bag, mosquito net, car cover, shoe, boot, sandal, slipper, sneaker, skate, lens, eyeglass, sunglass, uniform, armor, ballistic vest, bulletproof vest, bombsuit, life jacket, firesuit, protective suit, spacesuit, pressure suit, baselayer, underwear, lounge wear, brassiere, hosiery, daywear, evening wear, formal wear, bridal wear, outerwear, earwear, eyewear, headwear, footwear, dance suit, swimsuit, diving suit, track suit, skiwear, snowwear, rainwear, beach wear, activewear, sportswear, vehicle, and building;
wherein said clothing and covering having feature selected from group consisting of fabric disclosure, washing instruction, brand label, printed label, label, sign, patch, badge, stamp, tag, hood, pocket, collar, collar stay, shirt stay, lapel, neckline, turtle neck, mock neck, crew neck, funnel neck, cowl neck, jewel neck, boat neck, single breast, double breast, placket, pleat, fold, roll, ruffle, bubble, crop, sleeve, raglan, tail, cuff, hem, fly, gusset, corset, cleavage, thread, fastener, closure, knob, latch, frame, panel, rail, notion, seam, twill, button, button hole, hole, vent, air vent, pom pom, tassel, strap, buckle, belt, band, elastic band, tape, resealable tape, velcro, hook, eye, zipper, rivet, grommet, snap, seal, pin, safety pin, screw, screw cap, cap, bottle cap, safety cap, stay tab, pull tab, lid, cork, staple, slide, clasp, tack, nail, clip, knot, loop, string, drawstring, whip, rod, toggle, suspender, rope, tie, tape, handle, strap, flap, hinge, lock, wheel, chain, wire, underwire, ring, heel, shoelace, shank, insole, outsole, crampon, cleat, stud, welt, tongue, aglet, glue, ribbon, cord, shoulder pad, pad, gauze, film, mesh, wool, foam, gel, corduroy, crochet, velvet, velveteen, velour, satin, lace, cable, pique, honeycomb, sequin, chiffon, fleece, flannel, felt, denim, gabardine, herringbone, paisley, shield, shade, cover, cloth, towel, terry, braid, twist, net, canvas, needlework, quilt, embroidery, tapestry, upholstery, pattern, print, dye, color, lens, glass, ornament, jewelry, brooch, cuff link, corrugated fiberboard, paperboard, covered elastic band, covered hood under collar, silicon pad, stone washing, non ironing, preshrunk, seamless, stitching, sewing, knitting, weaving, quilting, crafting, tailoring, molding, shaping, cutting, opening, trimming, piping, beading, webbing, facing, backing, lining, filing, layering, pressure, oxygen, spray, pump, wicking reed, candle, wax, wick, fragrance, aroma, deodorant, moisturizer, cleanser, glow, filter, plant, coating, paint, polish, solar panel, drainage, slope, architecture, design, construction, electrical, plumbing, telecommunication, heating, ventilation, air conditioning, water, gas, lighting, escalator, elevator, blister pack, skin pack, and child resistant packaging;
wherein said clothing and covering including category selected from group consisting of rainwear, snowwear, skiwear, hospital, military, police, firefighting, lifeguard, space, flight, diving, protection wear, uniform, cleaning, kitchen, bathroom, bedding, brassiere, hosiery, eyewear, head wear, underwear, lounge wear, daywear, eveningwear, couture, casual wear, business wear, formal wear, bridal wear, beachwear, outerwear, footwear, activewear, sportswear, outdoor, towel, cloth, pad, cover, shade, shield, screen, blind, bandage, bag, box, bottle, container, food container, drink container, cosmetics container, fragrance container, moisturizer container, cleanser container, soap dispenser, luggage, curtain, carpet, door, window, wall, ceiling, floor, car window, window covering, floor covering, wall covering, ceiling covering, roof covering, solar panel, furniture, air filter, umbrella, lens, eyeglass, sunglass, computer, cell phone, vehicle, vehicle cover, and building;
wherein said at least one layer comprising on surface selected from group consisting of color, pattern, figure, drawing, writing, sign, label, logo, and advertisement;
wherein said at least one layer is selected from group consisting of disposable, replaceable, washable, and reusable;
wherein said at least one fabric is selected from group consisting of waterproof fabric, synthetic fabric, natural fabric, and elastic fabric; and
wherein said at least one function is selected from group consisting of wearing on body, fashion display, preventing snow, preventing wind, preventing sun, preventing dust and dirt, support, comfort, performance, protection, insulation, warmth, coverage, storage, containment, drying, wiping, drainage, security, housing, water flotation, thermal insulation, heat resistance, bullet proofing, sun screening, non ironing, breasts shaping, arch support, seamless footwear, bandaging, cover polarizing, pressure addition, cover planting, electricity generation, food storage with coated paper, odor absorption, aromatherapy, moisturizing, cleansing, dust trapping, glow-in-the-dark, water and leakage proofing, and ingredient addition.

2. The clothing and covering system of claim 1 wherein said at least one layer has addition of at least one ingredient selected from group consisting of flavoring ingredient, cleansing ingredient, nutritional ingredient, and health ingredient; wherein said at least one ingredient is delivered without using adhesive on skin; said at least one ingredient is delivered by using adhesive on skin; said at least one ingredient can be delivered to a large surface area of body; said at least one ingredient is delivered to specific part of body; said at least one ingredient is added to said at least one layer selected from group consisting of specific section of said at least one layer, and entire said at least one layer; said at least one ingredient is in form selected from group consisting of extract, juice, sauce, liquid, water, oil, alcohol, lotion, gel, wax, jelly, paste, powder, and small pieces; said at least one ingredient is added to said at least one layer by method selected from group consisting of imbedded, coated, sewn, and glued; and said at least one ingredient is contained in container with said at least one layer.

3. The clothing and covering system of claim 1 wherein said at least one fabric has addition of at least one ingredient selected from group consisting of flavoring ingredient, cleansing ingredient, nutritional ingredient, and health ingredient; said at least one ingredient is in form selected from group consisting of extract, juice, sauce, liquid, water, oil, alcohol, lotion, gel, wax, jelly, paste, powder, and small pieces; and said at least one ingredient is added to said at least one fabric by method selected from group consisting of imbedded, coated, sewn, and glued.

4. The clothing and covering system of claim 1 wherein said at least one fabric comprises waterproof fabric selected from group consisting of natural fabric, and synthetic fabric; said natural fabric includes shell, pearl, mineral, metal, rubber, resin, and wax; said synthetic fabric includes plastic, bio-plastic, latex, nylon, polyester, rayon, acrylic, acetate, lastex, kevlar, gore-tex, polyvinyl chloride, polyurethane, silicone elastomer, fluoropolymers, polyamide, polypropylene, polyethylene, polyethylene terephthalate, microfiber, polymer, fiberglass, glass, concrete, cement, brick, stucco, plaster, paint, resin, geopolymer, geotextile, ultrasuede, silicone, olefin, ingeo, lurex, nomex, marian, viscose, aramid, mineral wax, petroleum wax, and synthetic wax; and said waterproof fabric can be interwoven with said elastic fabric for clothing stretchability.

5. The clothing and covering system of claim 1 wherein said at least one fabric comprises synthetic fabric selected from group consisting of plastic, bio-plastic, latex, nylon, polyester, rayon, acrylic, modacrylic, acetate, lastex, kevlar, gore-tex, polyvinyl chloride, polyurethane, silicone elastomer, fluoropolymers, polyamide, polypropylene, polyethylene, polyethylene terephthalate, styrofoam, microfiber, polymer, fiberglass, glass, concrete, cement, brick, stucco, plaster, paint, resin, geopolymer, geotextile, ultrasuede, silicone, olefin, ingeo, lurex, nomex, marian, viscose, aramid, mineral wax, petroleum wax, and synthetic wax; and said synthetic fabric can be interwoven with said elastic fabric for clothing stretchability.

6. The clothing and covering system of claim 1 wherein said at least one fabric comprises natural fabric selected from group consisting of plant fiber, vegetable fiber, wood fiber, animal fiber, metallic fiber, carbon fiber, and mineral fiber, including cotton, silk, linen, flax, jute, sisal, ramie, tencel, wool, cashmere, alpaca, angora, llama, mohair, pashmina, camel hair, spider silk, bamboo, hemp, wood, paper, down, feather, leather, suede, shearling, fur, skin, hair, bone, horn, shell, pearl, mineral, stone, rock, soil, sand, clay, metal, rubber, resin, plant wax, and animal wax; said natural fabric has ability to absorb moisture and is comfortable to wear; said natural fabric can be interwoven with said synthetic fabric for clothing durability; said natural fabric can be interwoven with said elastic fabric for clothing stretchability; and said natural fabric can be interwoven with both said synthetic fabric and said elastic fabric.

7. The clothing and covering system of claim 1 wherein said at least one fabric comprises elastic fabric selected from group consisting of spandex, elastane, and lycra; and said elastic fabric has ability to stretch.

8. The clothing and covering system of claim 1 wherein said at least one function comprises water flotation which provides buoyancy for flotation in water by air inflation; and fabric used for said air inflation includes two layers of bonded polyurethane.

9. The clothing and covering system of claim 1 wherein said at least one function comprises thermal insulation which maintains temperature of content to stay cold and stay hot; and said thermal insulation using fabric selected from group consisting of PET film, styrofoam, polyethylene, polyurethane, and polypropylene.

10. The clothing and covering system of claim 1 wherein said at least one function comprises heat resistance which provides insulation from heat and fire; and said heat resistance using fabric selected from group consisting of marian, fiberglass, and modacrylic.

11. The clothing and covering system of claim 1 wherein said at least one function comprises bullet proofing which provides capability of stopping a bullet and similar high velocity projectile; and said bullet proofing using fabric selected from group consisting of kevlar, lexan, polyethylene, aramid, ceramic, metal, and carbon fiber composite.

12. The clothing and covering system of claim 1 wherein said at least one function comprises sun screening which provides sun protection, and sun protective clothing is produced from a fabric rated for its level of ultraviolet protection; and said sun screening using fabric selected from group consisting of fabric with a novel weave structure and denier, and fabric pre-treated with ultraviolet inhibiting ingredient including dye, titanium dioxide, and zinc oxide.

13. The clothing and covering system of claim 1 wherein said at least one function comprises non ironing with coating for permanent press which suppresses wrinkles and creates a smooth appearance without ironing; and said permanent press using chemical selected from group consisting of polytetrafluoroethylene, formaldehyde, urea-formaldehyde derivative, and urea derivative.

14. The clothing and covering system of claim 1 wherein said at least one function comprises breasts shaping which provides breasts with shaping of fit and size by method selected from group consisting of using bra underwire and padding; said bra underwire uses a thin, semi-circular strip of rigid material fitted inside bra fabric under each cup, and material for said bra underwire includes metal, plastic, and resin; and said padding uses a pad for each cup, and material for said padding includes natural fabric, synthetic fabric, and silicon gel.

15. The clothing and covering system of claim 1 wherein said at least one function comprises arch support which provides support for arch of foot by method selected from group consisting of placing a piece of material on top of insole of shoe having molded form fitting arch of foot, and making insole of shoe with material having molded form fitting arch of foot; and said material is selected from group consisting of natural fabric, synthetic fabric, plastic, bio-plastic, rubber, and silicon gel.

16. The clothing and covering system of claim 1 wherein said at least one function comprises seamless footwear which provides comfort for foot by using sock like seamless material in one piece to wrap foot for footwear; said seamless footwear is selected from group consisting of single layer, multiple layer, inner layer, middle layer, and outer layer; said footwear is selected from group consisting of shoe, boot, slipper, sneaker, skate, ski footwear, swim footwear, and diving footwear; said seamless material can be imbedded with material selected from group consisting of activated charcoal, baking soda, and magnets; said seamless material is selected from group consisting of disposable, replaceable, and reusable; and said seamless material is selected from group consisting of natural fabric, synthetic fabric, plastic, bio-plastic, rubber, and silicon gel.

17. The clothing and covering system of claim 1 wherein said at least one function comprises bandaging which provides support and dressing to specific part of body by method selected from group consisting of wrapping dressing without adhesive, wrapping dressing with adhesive, wrapping splint without adhesive, and wrapping splint with adhesive; said dressing is selected from group consisting of cloth, gauze, film, gel, foam, hydrocolloid, alginate, hydrogel, and polysaccharide paste, granule, and bead; and said splint is selected from group consisting of fiberglass, and orthoglass.

18. The clothing and covering system of claim 1 wherein said at least one function comprises cover polarizing which provides reduction of glare by using polarizer and polarizing filter; said cover polarizing is for cover selected from group consisting of eyewear, window, car window, door, shade, shield, screen, and curtain; and said cover polarizing using fabric selected from group consisting of synthetic plastic sheet including polyvinyl alcohol polymer impregnated with iodine, and aligned polyvinylene chains.

19. The clothing and covering system of claim 1 wherein said at least one function comprises pressure addition which provides suit wearer with pressurized oxygen within sealed suit; and said pressure addition is useful for suit selected from group consisting of space suit, flight suit, diving suit, ski suit, and pressure suit.

20. The clothing and covering system of claim 1 wherein said at least one function comprises cover planting which provides cover with planting; said cover planting is for cover selected from group consisting of wall, floor, ceiling, roof, window, door, shade, shield, screen, curtain, and carpet; said cover can be partially and completely covered by irrigated living plant; said cover planting is selected from group consisting of loose medium, mat medium, and structural medium; and said cover planting can provide environmental, social, and economic benefits.

21. The clothing and covering system of claim 1 wherein said at least one function comprises electricity generation which provides generation of electricity with installation of solar panels; said solar panels are installed on top of said clothing and covering, including roof, wall, building, and vehicle.

22. The clothing and covering system of claim 1 wherein said at least one function comprises food storage with coated paper which provides paper container and carton with ability of food storage; said food storage with coated paper can store liquid food and dry food; and said paper container and carton using coating selected from group consisting of plant wax, animal wax, beeswax, carnauba wax, paraffin wax, polyethylene, aluminum foil, and bio-plastic.

23. The clothing and covering system of claim 1 wherein said at least one function comprises odor absorption which provides absorption of odor from atmosphere; said odor absorption using container storing material selected from group consisting of activated charcoal, baking soda, zeolite, silica gel, oxidizing agent, air sanitizer, antiseptic, deodorant, and disinfectant; said material in form selected from group consisting of solid powder, solid particles, liquid, gel, wax, and jelly; said container selected from group consisting of cloth, paper, plastic, glass, and metal; said container in form selected from group consisting of porous container, and non porous container with top opening; said porous container in form selected from group consisting of box, bottle, and bag; and said box having edge supporting perimeter frame and porous layer on one to six sides for surface area exposure of enclosed said material.

24. The clothing and covering system of claim 1 wherein said at least one function comprises aromatherapy which provides treatment and prevention of disease by use of fragrant oil, fragrant water, fragrant alcohol, fragrant wax, and fragranced material; said aromatherapy having mode of application selected from group consisting of aerial diffusion, direct inhalation, and topical application; said aerial diffusion is by method selected from group consisting of vaporization, heat application, use of spray bottle, use of wicking reed, use of fragrant drawer paper and drawer cloth, use of fragrant botanical in porous container and cloth container, and burning of fragrant candle; said direct inhalation is by method selected from group consisting of inhaling from small container with said fragrant liquid, and using fragranced face mask; and said topical application is by method consisting of use of said clothing and covering, including spray bottle, pump bottle, container, and dressing.

25. The clothing and covering system of claim 1 wherein said at least one function comprises moisturizing which provides moisturizer selected from group consisting of plant oil, vegetable oil, mineral oil, jojoba oil, vegetable fat, animal fat, plant wax, animal wax, beeswax, carnauba wax, paraffin wax, aloe vera, petroleum jelly, collagen, snail slime, and antioxidant; said moisturizing having mode of application consisting of topical application; and said topical application is by method consisting of use of said clothing and covering, including spray bottle, pump bottle, container, and dressing.

26. The clothing and covering system of claim 1 wherein said at least one function comprises cleansing which provides cleanser by use of cleansing ingredient selected from group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, benzoyl peroxide, resorcinol, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium tetrachlorohydrex gly, activated charcoal, zeolite, silica gel, oxidizing agent, air sanitizer, antiseptic, deodorant, and disinfectant; said cleansing having mode of application consisting of topical application, wiping, and washing; and said mode of application is by method consisting of use of said clothing and covering, including spray bottle, pump bottle, container, and dressing.

27. The clothing and covering system of claim 1 wherein said at least one function comprises dust trapping which provides the ability to trap dust particles; said dust trapping is by method consisting of use of said clothing and covering, including dustcloth, air filter, screen, and face mask; said dust trapping using material selected from group consisting of tack, wax, oil, polyester, paper, and cloth; and said material is in form selected from group consisting of mesh, woven, pleated, layered, and textured.

28. The clothing and covering system of claim 1 wherein said at least one function comprises glow-in-the-dark which provides the ability to glow in the dark; said glow-in-the-dark is selected from group consisting of phosphorescence, and fluorescence; said phosphorescence using material selected from group consisting of zinc sulfide, and strontium aluminate; and said fluorescence using material selected from group consisting of vitamin B2, quinine, pyranine, gemstone, and mineral.

29. The clothing and covering system of claim 1 wherein said at least one function comprises water and leakage proofing which controls and prevents both leakage of discharge and secretion of body, and leakage of rain and water; said water and leakage proofing can be accomplished by leakproof clothing and covering; said leakproof clothing and covering comprise said at least one fabric selected from group consisting of said waterproof fabric, said synthetic fabric, said natural fabric, and said elastic fabric; said waterproof fabric of 1st kind is not interwoven with other fabric; said waterproof fabric of 2nd kind is interwoven with said elastic fabric; said natural fabric of 1st kind is not interwoven with other fabric; said natural fabric of 2nd kind is interwoven with said synthetic fabric; said natural fabric of 3rd kind is interwoven with said elastic fabric; and said natural fabric of 4th kind is interwoven with both said synthetic fabric and said elastic fabric.

30. The clothing and covering system of claim 1 wherein said at least one function comprises ingredient addition which provides addition of at least one ingredient selected from group consisting of flavoring ingredient, cleansing ingredient, nutritional ingredient, and health ingredient.

31. The clothing and covering system of claim 29 wherein said leakproof clothing and covering can be used in conjunction with disposable including sanitary pad, sanitary napkin, panty liner, tampon, incontinence pad, diaper, and underarm liner; said disposable comprises material selected from group consisting of adhesive, resealable tape, elasticised waist band, natural fabric, synthetic fabric, superabsorbent polymer, oil, gel, wax, jelly, lotion, and fragrance; and said disposable can be placed and glued to specific area of said leakproof clothing and covering, including underarm area, foot area, hand area, neck area, head area, crotch area, groin area, and genital area.

32. The clothing and covering system of claim 29 wherein said leakproof clothing and covering has 26 kinds.

33. The clothing and covering system of claim 29 wherein said leakproof clothing and covering of 1st kind is created with a single layer of said waterproof fabric, and wearer can have option to wear a garment underneath said leakproof clothing and covering; and said garment can be made of said natural fabric.

34. The clothing and covering system of claim 29 wherein said leakproof clothing and covering of 2nd kind is created with a single layer of said natural fabric, with an outer coating of said waterproof fabric, and an inner side has said natural fabric.

35. The clothing and covering system of claim 29 wherein said leakproof clothing and covering of 3rd kind is created with a single layer of said natural fabric, with an inner coating of said waterproof fabric, and an outer side has said natural fabric.

36. The clothing and covering system of claim 29 wherein said leakproof clothing and covering of 4th kind is created with a single layer of said natural fabric, with an inner coating of said waterproof fabric, and an outer coating of said waterproof fabric.

37. The clothing and covering system of claim 29 wherein said leakproof clothing and covering of 5th kind is created by having 2 layers, with an inner layer of said natural fabric, and an outer layer of said waterproof fabric.

38. The clothing and covering system of claim 29 wherein said leakproof clothing and covering of 6th kind is created by having 2 layers, with an outer layer of said natural fabric, and an inner layer of said waterproof fabric.

39. The clothing and covering system of claim 29 wherein said leakproof clothing and covering of 7th kind is created with a single layer of said natural fabric, having section made of said waterproof fabric as an outer coating, and an inner side has said natural fabric; and said section includes underarm area, crotch area, groin area, genital area, upper torso of the body, lower torso of the body, trunk of the body, feet, hand, neck, and head.

40. The clothing and covering system of claim 29 wherein said leakproof clothing and covering of 8th kind is created with a single layer of said natural fabric, having section made of said waterproof fabric as an inner coating, and an outer side has said natural fabric; and said section includes underarm area, crotch area, groin area, genital area, upper torso of the body, lower torso of the body, trunk of the body, feet, hand, neck, and head.

41. The clothing and covering system of claim 29 wherein said leakproof clothing and covering of 9th kind is created with a single layer of said natural fabric, having section made of said waterproof fabric as an inner coating, and an outer coating of said waterproof fabric; and said section includes underarm area, crotch area, groin area, genital area, upper torso of the body, lower torso of the body, trunk of the body, feet, hand, neck, and head.

42. The clothing and covering system of claim 29 wherein said leakproof clothing and covering of 10th kind is created with a single layer of said at least one fabric, having section made of said waterproof fabric, and other area of single layer made of said natural fabric; and said section includes underarm area, crotch area, groin area, genital area, upper torso of the body, lower torso of the body, trunk of the body, feet, hand, neck, and head.

43. The clothing and covering system of claim 29 wherein said leakproof clothing and covering of 11th kind is created by having 2 layers, having section made of said waterproof fabric as an outer layer, and an inner layer of said natural fabric; and said section includes underarm area, crotch area, groin area, genital area, upper torso of the body, lower torso of the body, trunk of the body, feet, hand, neck, and head.

44. The clothing and covering system of claim 29 wherein said leakproof clothing and covering of 12th kind is created by having 2 layers, having section made of said waterproof fabric as an inner layer, and an outer layer of said natural fabric; and said section includes underarm area, crotch area, groin area, genital area, upper torso of the body, lower torso of the body, trunk of the body, feet, hand, neck, and head.

45. The clothing and covering system of claim 29 wherein said leakproof clothing and covering of 13th kind is created by having a middle layer of said waterproof fabric between 2 layers of said natural fabric.

46. The clothing and covering system of claim 29 wherein said leakproof clothing and covering of 14th kind is created by having a middle layer of said waterproof fabric between 2 layers of said waterproof fabric.

47. The clothing and covering system of claim 29 wherein said leakproof clothing and covering of 15th kind is created by having a middle layer of said waterproof fabric between 2 layers, with an inner layer of said natural fabric, and an outer layer of said waterproof fabric.

48. The clothing and covering system of claim 29 wherein said leakproof clothing and covering of 16th kind is created by having a middle layer of said waterproof fabric between 2 layers, with an outer layer of said natural fabric, and an inner layer of said waterproof fabric.

49. The clothing and covering system of claim 29 wherein said leakproof clothing and covering of 17th kind is created by having a middle layer of said natural fabric between 2 layers of said waterproof fabric.

50. The clothing and covering system of claim 29 wherein said leakproof clothing and covering of 18th kind is created by having a middle layer of said natural fabric between 2 layers of said natural fabric, with an outer layer having an outer coating of said waterproof fabric.

51. The clothing and covering system of claim 29 wherein said leakproof clothing and covering of 19th kind is created by having a middle layer of said natural fabric between 2 layers of said natural fabric, with an inner layer having an outer coating of said waterproof fabric.

52. The clothing and covering system of claim 29 wherein said leakproof clothing and covering of 20th kind is created by having a middle layer of said natural fabric between 2 layers of said natural fabric, with an outer layer having an outer coating of said waterproof fabric, and an inner layer having an outer coating of said waterproof fabric.

53. The clothing and covering system of claim 29 wherein said leakproof clothing and covering of 21st kind is created by having a middle layer of said natural fabric between 2 layers, with an inner layer of said natural fabric, and an outer layer of said waterproof fabric.

54. The clothing and covering system of claim 29 wherein said leakproof clothing and covering of 22nd kind is created by having a middle layer of said natural fabric between 2 layers, with an outer layer of said natural fabric, and an inner layer of said waterproof fabric.

55. The clothing and covering system of claim 29 wherein said leakproof clothing and covering of 23rd kind is created by having said at least one layer of said waterproof fabric.

56. The clothing and covering system of claim 29 wherein said leakproof clothing and covering of 24th kind is created by having at least one coating of said waterproof fabric.

57. The clothing and covering system of claim 29 wherein said leakproof clothing and covering of 25th kind is created by having at least one section of said at least one layer made of said waterproof fabric; and said section includes underarm area, crotch area, groin area, genital area, upper torso of the body, lower torso of the body, trunk of the body, feet, hand, neck, and head.

58. The clothing and covering system of claim 29 wherein said leakproof clothing and covering of 26th kind is created by having at least one section of said at least one layer having at least one coating of said waterproof fabric; and said section includes underarm area, crotch area, groin area, genital area, upper torso of the body, lower torso of the body, trunk of the body, feet, hand, neck, and head.

59. The clothing and covering system of claim 30 wherein said flavoring ingredient can provide smell of flavor, fresh scent, aroma, and fragrance.

60. The clothing and covering system of claim 30 wherein said flavoring ingredient is selected from group consisting of natural flavor, artificial flavor, fruit flavor, plant flavor, herb flavor, tea flavor, root flavor, flower flavor, flower bud flavor, pollen flavor, leaf flavor, stem flavor, stem shoot flavor, bulb flavor, nut flavor, seed flavor, vegetable flavor, bean flavor, cereal grain flavor, plant oil flavor, vegetable oil flavor, vegetable fat flavor, vegetable skin flavor, and fruit skin flavor, including artificial sweetener, salt, vinegar, sugar, syrup, honey, milk, yogurt, butter, margarine, cream, sour cream, peanut butter, butterscotch, caramel, cheese, chocolate, coffee, roasted cereal grain beverage, roasted nut and seed beverage, roasted bean beverage, malted drink, flower tea, root tea, herbal tea, mushroom tea, green tea, oolong tea, black tea, white tea, yellow tea, earl grey, darjeeling, hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litsea cubeba, marjoram, melaleuca, mountain savory, mugwort, myrrh, myrtle, neroli, nutmeg, orris, palo santo, petitgrain, pine, ravensara, red cedar, rosewood, sage, sassafras, schisandra, spikenard, spruce, tea tree, tsuga, valerian, yarrow, ylang ylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiño, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain
amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, mint, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff.

61. The clothing and covering system of claim 30 wherein said cleansing ingredient can eliminate bacteria and odor, and said clothing and covering with said cleansing ingredient is less likely to contaminate and emit odor.

62. The clothing and covering system of claim 30 wherein said cleansing ingredient is selected from group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, benzoyl peroxide, resorcinol, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium
tetrachlorohydrex gly, activated charcoal, zeolite, silica gel, oxidizing agent, air sanitizer, antiseptic, deodorant, and disinfectant.

63. The clothing and covering system of claim 30 wherein said nutritional ingredient can provide essential nutrient.

64. The clothing and covering system of claim 30 wherein said nutritional ingredient is selected from group consisting of essential nutrient, including vitamin, dietary mineral, amino acid, and fatty acid; said vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K; said dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium; said amino acid includes histidine, isoleucine, leucine, lysine, methionine, cysteine, phenylalanine, tyrosine, threonine, tryptophan, and valine; and said fatty acid includes alpha-linoleic acid, and linoleic acid.

65. The clothing and covering system of claim 30 wherein said health ingredient can provide health benefit.

66. The clothing and covering system of claim 30 wherein said health ingredient is selected from group consisting of magnet, and medication; said magnet produces magnetic therapy, with magnetic energy enabling people to relieve pain, reduce inflammation, produce an alkaline effect, fight infection, support healing, reduce fluid retention, increase cellular oxygen, encourage deep restorative sleep, produce a bright mental effect, reduce fatty deposit, establish healing polarity, stimulate melatonin production, and normalize natural alkaline pH; and said medication can be absorbed into skin and bloodstream.

67. The clothing and covering system of claim 66 wherein said magnet is in form of small round discs about 1 to 3 millimeters in thickness and about 5 to 30 millimeters in diameter; said magnet varying from about 100 Gauss to 2,500 Gauss as actual reading with a Gauss meter; said magnet having North Pole facing body of wearer; said magnet is selected from group consisting of ceramic magnet, neodymium magnet, samarium cobalt magnet, and flexible rubber magnet; and said magnet is added by method selected from group consisting of imbedded, coated, sewn, and glued.

68. The clothing and covering system of claim 66 wherein said medication is applied in order to deliver medication into skin; said medication is applied in order to deliver medication into bloodstream; said medication is delivered without using adhesive on skin; said medication is delivered by using adhesive on skin; said medication can be delivered to a large surface area of body; said medication is delivered to specific part of body; said medication is applied to area selected from group consisting of specific section of said clothing and covering, and entire said clothing and covering; said medication is in form selected from group consisting of extract, liquid, water, oil, alcohol, lotion, gel, wax, jelly, paste, powder, and small pieces; and said medication is applied by method selected from group consisting of imbedded, coated, sewn, and glued.

69. The clothing and covering system of claim 1 is created for animal, human, infant, children, and adult; and in all sizes.
